(19) United States
(12) Patent Application Publication
(10) Pub. No.: US 2014/0127037 A1
(43) Pub. Date: May 8, 2014

(54) FLUID SUPPLY APPARATUS

(71) Applicant: SEIKO EPSON CORPORATION, Tokyo (JP)

(72) Inventors: Kazuaki UCHIDA, Nagano-ken (JP); Takeshi SETO, Tokyo-to (JP); Takahiro MATSUZAKI, Shiojiri-shi (JP); Atsuya HIRABAYASHI, Chino-shi (JP)

(73) Assignee: SEIKO EPSON CORPORATION, Tokyo (JP)

(21) Appl. No.: 14/072,260

(22) Filed: Nov. 5, 2013

(30) Foreign Application Priority Data
Nov. 7, 2012 (JP) 2012-244984

(51) Int. Cl. F04B 11/00; F04B 49/22; A61M 5/172; F04B 7/00

(52) U.S. Cl. CPC F04B 11/005; F04B 7/0076; F04B 49/225; A61M 5/172
USPC 417/53; 417/279; 417/62; 604/67

(57) ABSTRACT

A fluid supply apparatus which supplies a fluid to a medical apparatus includes: a pump mechanism including a first pump capable of carrying out a intake operation of a fluid and a feeding operation of the fluid, and a second pump capable of alternately carrying out a intake operation of the fluid and a feeding operation of the fluid; a flow passage which includes an elastic member and which communicates with the pumps and supplies the fluid to the medical apparatus; a pressure fluctuation detecting unit capable of detecting fluctuation in internal pressure in the flow passage; and a flow passage deforming unit which deforms the flow passage according to the fluctuation in the internal pressure.

This application claims priority to Japanese Patent Application No. 2012-244984 filed on Nov. 7, 2012. The entire disclosure of the Japanese Patent Application No. 2012-244984 is hereby incorporated herein by reference.

### BACKGROUND

1. Technical Field

The present invention relates to a technique for supplying a fluid to a medical apparatus.

2. Related Art

According to a related art, for example, a technique disclosed in JP-A-9-264261 is known as a technique for stably feeding a fluid. JP-A-9-264261 discloses a technique in which when one of two plunger pumps is carrying out an discharge process at a predetermined discharge pressure, the other stands by in the state of pre-pressurizing the fluid, and when the discharge pressure of the one plunger pump begins to fall, the other starts a pressurization and discharge process of the fluid and raises the pressure to a target discharge pressure within a short time, thus carrying out discharge continuously.

However, in the technique of JP-A-9-264261, a periodic pulsating flow is generated when the discharge is switched between the two plunger pumps. For example, in the case where the technique is applied to a fluid supply apparatus which feeds a fluid to a water jet knife as a medical apparatus, a problem is pointed out that a pulsating flow is generated in the fluid ejected from the water jet knife, which is undesirable to the operation of the water jet knife. Also, various other issues are pointed such as reduction in size of the device, reduction in cost, resource saving, easier manufacturing, and improvement in user-friendliness. Such problems are equally seen in devices for supplying a fluid not only to a water jet knife but also to other medical apparatuses.

### SUMMARY

An advantage of some aspects of the invention is to solve at least a part of the problems described above, and the invention can be implemented as the following aspects.

(1) An aspect of the invention provides a fluid supply apparatus which supplies a fluid to a medical apparatus. The fluid supply apparatus includes: a pump mechanism having a first pump capable of alternately carrying out a intake operation of the fluid and a feeding operation of the fluid, and a second pump capable of alternately carrying out a intake operation of the fluid and a feeding operation of the fluid; a flow passage which includes an elastic member and which communicates with the pumps and supplies the fluid to the medical apparatus; a pressure fluctuation detecting unit capable of detecting fluctuation in internal pressure in the flow passage; and a flow passage deforming unit which deforms a part of the elastic member according to the fluctuation in the internal pressure and thus restrains pressure fluctuation of the fluid supplied to the medical apparatus. According to the fluid supply apparatus of this embodiment, by deforming the flow passage according to the fluctuation in the internal pressure in the flow passage and thereby restraining pressure fluctuation of the fluid supplied to the medical apparatus, fluctuation in the flow rate of the fluid supplied to the medical apparatus can be restrained.

(2) The fluid supply apparatus according to the aspect described above may be configured such that the pressure fluctuation detecting unit detects fluctuation in the internal pressure based on a displacement of an outer shape of the flow passage. According to the fluid supply apparatus of this aspect, the fluctuation in the pressure can be detected based on the appearance of the flow passage. For example, a displacement of the outer shape can be measured in a non-contact manner.

(3) The fluid supply apparatus according to the aspect described above may be configured such that the pressure fluctuation detecting unit is installed at a predetermined position on an outer wall surface of the flow passage and detects a force received from the flow passage and detects fluctuation in the internal pressure based on a result of the detection. According to the fluid supply apparatus of this aspect, since a force that is directly received from the flow passage is detected, the fluctuation in the internal pressure can be detected easily and accurately.

(4) The fluid supply apparatus according to the aspect described above may be configured such that the pressure fluctuation detecting unit receives, as the force received from the flow passage, a force due to the internal pressure in the flow passage and a force by the flow passage deforming unit to deform the flow passage, acquires the force due to the internal pressure in the flow passage based on the detected force received from the flow passage and the force by the flow passage deforming unit to deform the flow passage, and detects fluctuation in the internal pressure. According to the fluid supply apparatus of this aspect, the pressure fluctuation detecting unit can be installed at a position on the flow passage where the force due to the internal pressure in the flow passage and the force by the flow passage deforming unit to deform the flow passage are received.

(5) The fluid supply apparatus according to the aspect described above may be configured such that the flow passage deforming unit has a piezoelectric element which deforms the flow passage, and the pressure fluctuation detecting unit detects a force received from the flow passage with the piezoelectric element. According to the fluid supply apparatus of this aspect, since the single piezoelectric element has the functions of the flow passage deforming unit and the pressure fluctuation detecting unit, a simplified structure and reduction in cost can be realized.

(6) The fluid supply apparatus according to the aspect described above may be configured such that the pressure fluctuation detecting unit is a strain gauge installed at a predetermined position on an outer wall surface of the flow passage. According to the fluid supply apparatus of this aspect, pressure fluctuation in the flow passage can be detected by a relatively simple method.

(7) The fluid supply apparatus according to the aspect described above may be configured such that the flow passage deforming unit is capable of blocking the flow passage by the deformation of the flow passage and thus stopping supply of the fluid. According to the fluid supply apparatus of this aspect, supply of the fluid can be stopped by the flow passage deforming unit.

(8) The fluid supply apparatus according to the aspect described above may be configured such that the medical apparatus is a therapeutic apparatus which ejects a fluid to a living body and thus treats the living body. According to the fluid supply apparatus of this aspect, the medical apparatus which ejects a fluid can be supplied with the fluid at a stable flow rate with little pressure fluctuation.

(9) Another aspect of the invention provides a fluid supply apparatus which supplies a fluid to a medical apparatus. The fluid supply apparatus includes: a pump mechanism having plural pumps capable of alternately carrying out a intake operation of the fluid and a feeding operation of the fluid; a flow passage which includes an elastic member and which communicates with the pumps and supplies the fluid to the medical apparatus; a pressure fluctuation detecting unit capable of detecting fluctuation in internal pressure in the flow passage; and a flow passage deforming unit which deforms a part of the elastic member according to the fluctuation in the internal pressure and thus restrains pressure fluctuation of the fluid supplied to the medical apparatus. According to the fluid supply apparatus of this embodiment, by deforming the flow passage according to the fluctuation in the internal pressure in the flow passage, pressure fluctuation of the fluid supplied to the medical apparatus is restrained. Consequently, fluctuation in the flow rate of the fluid supplied to the medical apparatus can be restrained.

The invention can be implemented in various embodiments. For example, the invention can be implemented in such forms as a water jet knife system, fluid supply system, fluid supply method, and pulsating flow control method.

### BRIEF DESCRIPTION OF THE DRAWINGS

The invention will be described with reference to the accompanying drawings, wherein like numbers reference like elements.

FIG. 1 is an explanatory view showing a water jet knife system.

FIG. 2 is an explanatory view showing supply of water by a fluid supply apparatus.

FIGS. 3A and 3B are explanatory views showing a flow passage deforming mechanism.

FIGS. 4A and 4B are explanatory views showing the structure of the flow passage deforming mechanism.

FIGS. 5A to 5C are explanatory views showing a pressure fluctuation detecting unit.

FIGS. 6A and 6B are explanatory views showing the operation of plunger pumps.

FIGS. 7A to 7C are explanatory views showing pressure fluctuation restraint carried out by a control unit.

FIG. 8 is an explanatory view showing the structure of a water jet knife.

FIG. 9 is an explanatory view showing a water jet knife system.

FIG. 10 is a vertical sectional view showing a flow passage deforming mechanism including a pressure fluctuation detecting unit.

FIGS. 11A and 11B are explanatory views showing a force received by the pressure fluctuation detecting unit.

FIGS. 12A and 12B are explanatory views illustrating a force due to flow passage deformation.

FIG. 13 is an explanatory view showing an example of Modification.

FIGS. 14A and 14B are explanatory views showing an example of Modification.

FIGS. 15A to 15C are explanatory views showing an example of Modification.

FIGS. 16A and 16B are explanatory views showing an example of a water jet knife that can be employed.

### DESCRIPTION OF EXEMPLARY EMBODIMENTS

### A. First Embodiment

### A1. System Configuration

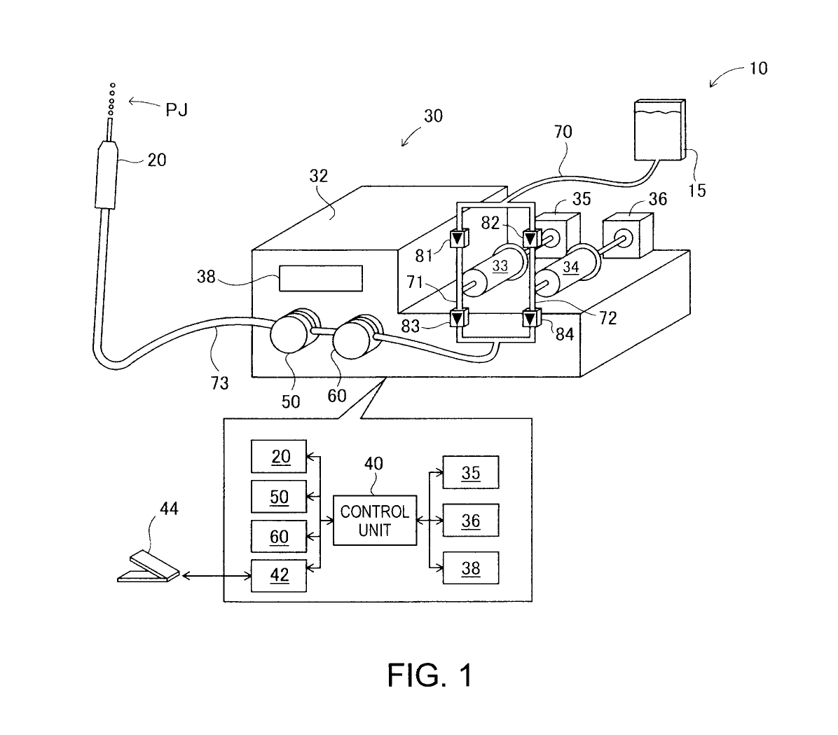

FIG. 1 is an explanatory view illustrating a water jet knife system 10 using a fluid supply apparatus as a first embodiment of the invention. A water jet knife is a kind of surgical knife and ejects a fluid at a high pressure to perform incision and excision with the discharge pressure. In this embodiment, sterilized water is employed as a fluid to be ejected.

The water jet knife system 10 has a water jet knife 20, a fluid supply apparatus 30 which supplies water to the water jet knife 20, and a fluid storage unit 15 which stores water to be supplied to the water jet knife 20. The water jet knife 20 has, inside itself, a mechanism which generates a pulse water flow using a piezoelectric element as a power source. In the water jet knife 20, the piezoelectric element is driven at a predetermined frequency to eject water supplied from the fluid supply apparatus 30 to outside as a pulsed high-pressure jet water flow (pulse jet PJ). The structure of the water jet knife 20 will be described in detail later.

The water jet knife 20, the fluid supply apparatus 30, and the fluid storage unit 15 are connected with each other via flow passages 70 to 73. Specifically, the fluid storage unit 15 is connected to one end of the flow passage 70. The other end of the flow passage 70 is connected to a diverging point between the flow passage 71 and the flow passage 72. The flow passage 71 is connected to a plunger pump 33 provided in the fluid supply apparatus 30. The flow passage 72 is connected to a plunger pump 34 provided in the fluid supply apparatus 30. The flow passage 71 and the flow passage 72 join together and are connected to the flow passage 73. The flow passage 73 is connected to the water jet knife 20.

The water in the fluid storage unit 15 is supplied to the water jet knife 20 via the flow passages 70 to 73 by the operation of the fluid supply apparatus 30. Check valves 81 to 84 are provided in the flow passages 71, 72. The water circulating through the flow passages 70 to 73 circulates only in one direction from the fluid storage unit 15 toward the water jet knife 20. The flow passages 70 to 73 are tubes made of polyvinyl chloride and are elastic. As the flow passage 70 to 73, elastic tubes made of silicone, thermoplastic elastomer or the like may also be employed.

The fluid supply apparatus 30 has a casing 32, plunger pumps 33, 34, pump drive units 35, 36, a display unit 38, a flow passage deforming mechanism 50, and a pressure fluctuation detecting unit 60. The plunger pumps 33, 34 carry out a intake operation to suck water from the fluid storage unit 15 and a feeding operation to feed the sucked water to the water jet knife 20. The pump drive units 35, 36 function as a power source for the plunger pumps 33, 34 to carry out the intake operation and the feeding operation. The pump drive units 35, 36 have a motor as a power source and thus realize the intake operation and the feeding operation by the plunger pumps 33, 34.

The display unit 38 is a display unit which displays various kinds of information about the supply of water, such as the amount of water supplied to the water jet knife 20 by the fluid supply apparatus 30, and the flow speed. The flow passage deforming mechanism 50 is a mechanism which holds the flow passage 73 therein and deforms the flow passage 73. The pressure fluctuation detecting unit 60 is a mechanism which detects fluctuation in internal pressure in the flow passage 73. The flow passage deforming mechanism 50 and the pressure fluctuation detecting unit 60 will be described in detail later.

As illustrated, the fluid supply apparatus 30 has a control unit 40 inside the casing 32. The control unit 40 is connected to the water jet knife 20, the pump drive units 35, 36, the display unit 38, an input/output IF 42, the flow passage deforming mechanism 50, and the pressure fluctuation detecting unit 60, and controls the operation of each device. A foot switch 44 for a user to operate discharge and stop of water from the water jet knife 20 is connected to the input/output IF 42. As the user operates the foot switch 44, the control unit 40 causes the water jet knife 20 and the fluid supply apparatus 30 (pump drive units 35, 36) to operate synchronously. Specifically, when the user uses the foot switch 44 to carry out an operation to eject a pulse jet PJ from the water jet knife 20, the control unit 40 drives the pump drive units 35, 36 and thus causes the water jet knife 20 to supply water, and the control unit 40 also controls the water jet knife 20 to eject the supplied water to outside as a pulse jet PJ.

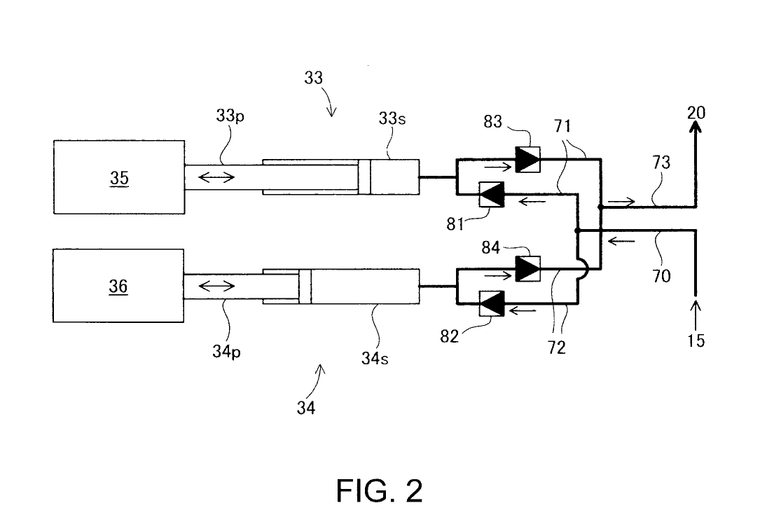

FIG. 2 is an explanatory view illustrating the supply of water by the fluid supply apparatus 30. FIG. 2 shows a configuration involved in the circulation of water, mainly around plunger pumps 33, 34. As illustrated, the plunger pump 33 has a syringe 33s and the plunger 33p. Similarly, the plunger pump 34 has a syringe 34s and a plunger 34p. The plunger 33p and the plunger 34p are attached to the pump drive unit 35 and the pump drive unit 36, respectively.

The pump drive units 35, 36 push and pull the plunger 33p and the plunger 34p, respectively, to cause plungers to reciprocate. As illustrated, an operation in which the plunger 33p (34p) is pushed into the syringe 33s (34s) is called a feeding operation of the plunger pump 33 (34). Meanwhile, an operation in which the plunger 33p (34p) is pulled out of the syringe 33s (34s) is called a intake operation of the plunger pump 33 (34). The plunger pumps 33, 34 function as displacement pumps with the power of the pump drive units 35, 36.

The flow passage 71 is connected to the plunger pump 33. The flow passage 72 is connected to the plunger pump 34. When the plunger pump 33 carries out the intake operation, the water in the fluid storage unit 15 is sucked into the syringe 33s via the flow passage 70, the flow passage 71, and the check valve 81. When the plunger pump 33 carries out the feeding operation, the water in the syringe 33s is fed to the water jet knife 20 via the check valve 83, the flow passage 71, and the flow passage 73. The intake operation and the feeding operation carried out by the plunger pump 34 are based on similar principles to the plunger pump 33 and therefore will not be described further in detail.

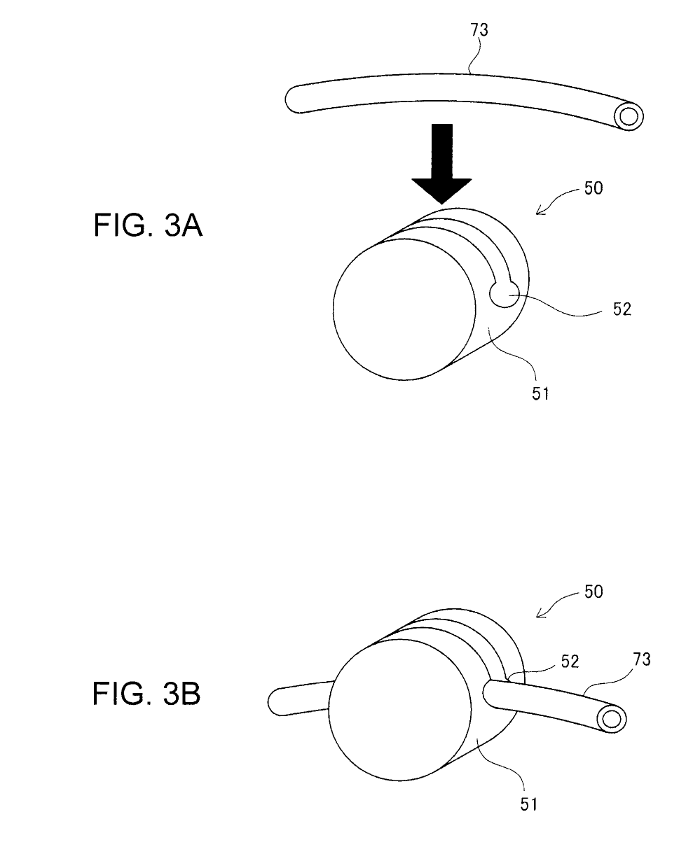

Next, the flow passage deforming mechanism 50 provided in the fluid supply apparatus 30 will be described. FIGS. 3A and 3B are explanatory views illustrating the flow passage deforming mechanism 50. The flow passage deforming mechanism 50 has a flow passage fixing portion 51 which can be observed from outside, and a pressing mechanism 53 (later described) formed inside the casing 32. FIGS. 3A and 3B show the flow passage fixing portion 51 which can be observed from outside. A groove portion 52 for holding the flow passage 73 therein is formed in the flow passage fixing portion 51. When using the water jet knife system 10, the user fits the flow passage 73 into the groove portion 52 as shown in FIGS. 3A and 3B.

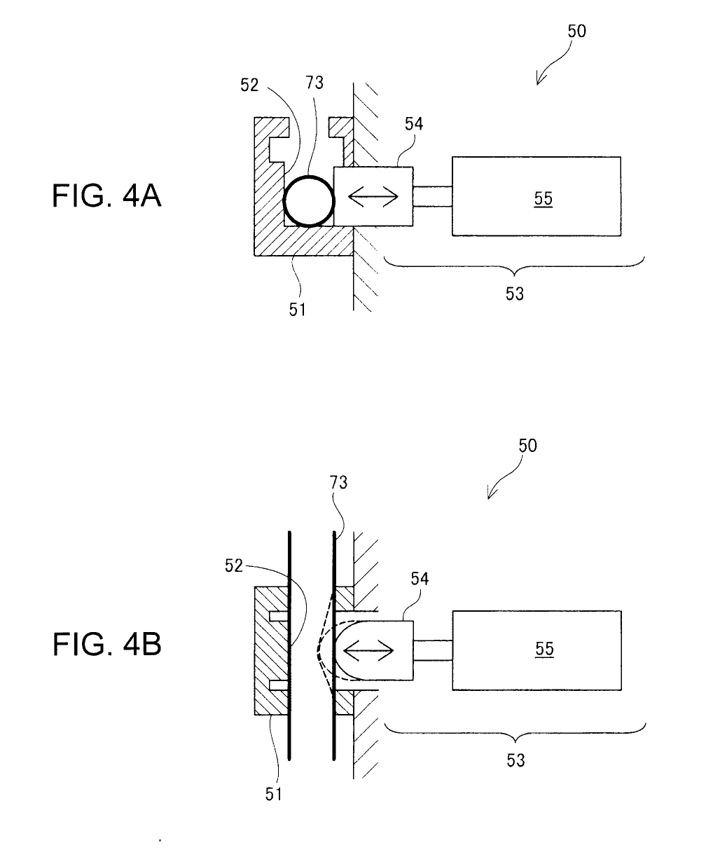

FIGS. 4A and 4B are explanatory views illustrating the structure of the flow passage deforming mechanism 50, including portions formed inside the casing 32. FIG. 4A is an explanatory view showing a vertical sectional structure of the flow passage deforming mechanism 50. FIG. 4B is an explanatory view showing a horizontal sectional structure of the flow passage deforming mechanism 50. The flow passage deforming mechanism 50 has the above flow passage fixing portion 51 and also has a pressing mechanism 53 inside the casing 32. The pressing mechanism 53 has a pressing portion 54 to press the flow passage 73, and a linear actuator 55 which applies a pressing force to the pressing portion 54. As illustrated, the linear actuator 55 is horizontally driven in a direction of pressing the flow passage 73 (hereinafter also referred to as a pressing direction). The horizontal driving of the linear actuator 55 causes the pressing portion 54 to press a wall surface of the flow passage 73. As shown in FIG. 4B, the surface of the pressing portion 54 that contacts the flow passage 73 is a curved surface and thus restrains damage to the flow passage 73 by the pressing.

It is desirable that the flow passage 73 is disposable. For example, disposing of the flow passage every treatment or every treatment procedure for one patient provides the fluid supply apparatus 30 with excellent hygiene. Moreover, since the flow passage 73 is disposed of, the wall surface of the flow passage 73 does not easily deteriorate. If the wall surface of the flow passage 73 is made of an elastic member, an expected elastic action can be achieved.

As shown in FIG. 4B, when the pressing mechanism 53 presses the flow passage 73, the flow passage 73 is locally deformed. When water is circulating in the flow passage 73, the pressure inside the flow passage 73 is increased by the pressing with the pressing mechanism 53. When water is supplied to the water jet knife 20 from the fluid supply apparatus 30, the control unit 40 drives the pressing mechanism 53 according to fluctuation in the pressure in the flow passage 73 detected by the pressure fluctuation detecting unit 60, and restrains the pressure fluctuation of the water supplied to the water jet knife 20 (pressure fluctuation restraint). The pressure fluctuation restraint carried out by the control unit 40 will be described in detail later.

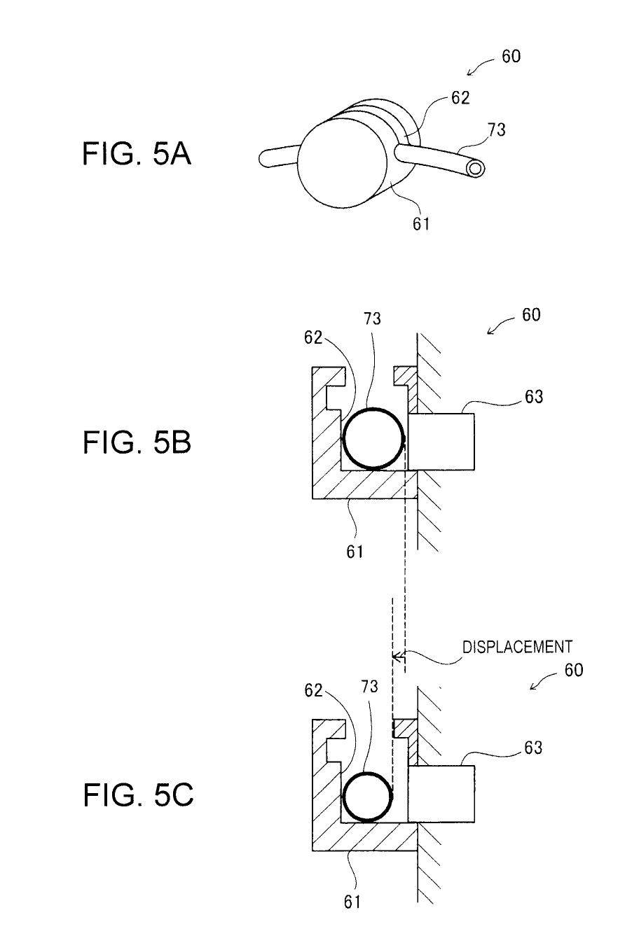

FIGS. 5A to 5C are explanatory views illustrating the pressure fluctuation detecting unit 60. The pressure fluctuation detecting unit 60 has a flow passage fixing portion 61 which can be observed from outside, and a laser displacement meter 63 formed inside the casing 32. FIG. 5A shows the flow passage fixing portion 61 which can be observed from outside. A groove portion 62 for holding the flow passage 73 therein is formed in the flow passage fixing portion 61. When using the water jet knife system 10, the user fits the flow passage 73 into the groove portion 62.

FIGS. 5B and 5C are explanatory views showing a vertical sectional structure of the pressure fluctuation detecting unit 60. The pressure fluctuation detecting unit 60 has the above flow passage fixing portion 61 and also has the laser displacement meter 63 inside the casing 32. The laser displacement meter 63 casts a semiconductor laser to the flow passage 73 and receives reflected light from the flow passage 73, thus measuring change in the width (diameter) of the flow passage 73. Since the flow passage 73 is elastic, its width (diameter) changes depending on internal pressure change. FIG. 5B shows the state where the internal pressure in the flow passage 73 is high. FIG. 5C shows the state where the internal pressure is low. As illustrated, when the internal pressure in the flow passage 73 changes, the distance between the laser displacement meter 63 and the flow passage 73 changes. The laser displacement meter 63 measures the width (diameter) of the flow passage 73, based on the change in the distance from the flow passage 73. The control unit 40 acquires fluctuation in the internal pressure (pressure fluctuation) in the flow passage 73, based on the width of the flow passage 73 measured by the laser displacement meter 63.

Specifically, the control unit 40 has a lookup table showing a correlation between the width (diameter) of the flow passage 73 and the internal pressure in the flow passage 73, enters the width (diameter) of the flow passage 73 measured by the laser displacement meter 63 into the lookup table, and acquires a corresponding internal pressure value. The control unit 40 acquires an internal pressure value at predetermined intervals and acquires pressure fluctuation based on the difference between preceding and subsequent values. The value of pressure fluctuation acquired by using the pressure fluctuation detecting unit 60 is used for the pressure fluctuation restraint carried out by the control unit 40. Hereinafter, the pressure fluctuation restraint carried out by the control unit 40 will be described.

### A2. Pressure Fluctuation Restraint

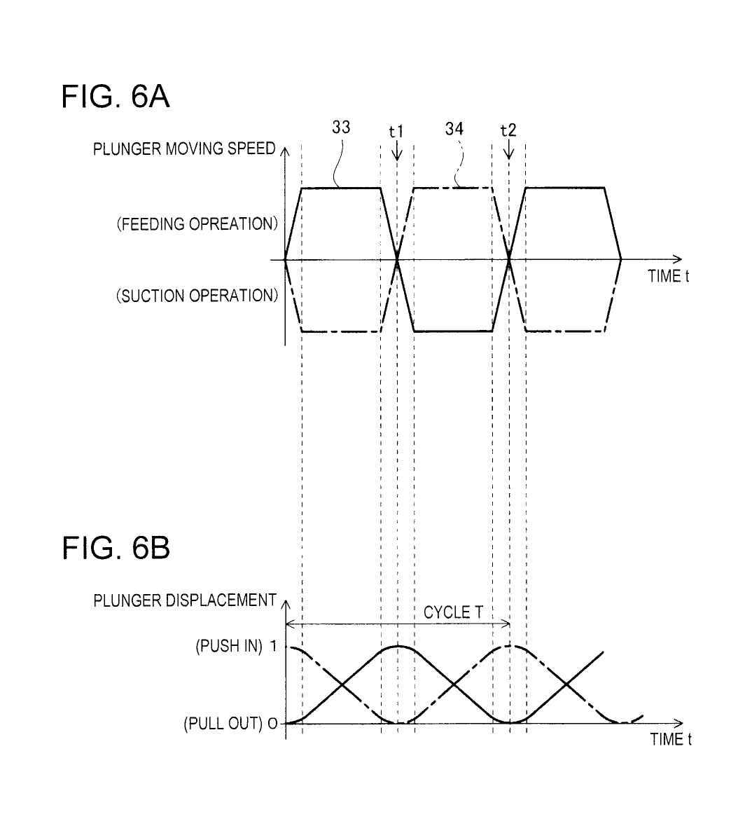

FIGS. 6A and 6B are explanatory views illustrating the operation of the plunger pumps 33, 34 under the control of the control unit 40 when the fluid supply apparatus 30 supplies water to the water jet knife 20. FIG. 6A shows the moving speed of the plungers 33p, 34p. The solid line on the graph shows the moving speed of the plunger 33p. The chain dotted line shows the moving speed of the plunger 34p. In the graph corresponding to each plunger pump (33, 34), the portion along values 0 and above on the vertical axis corresponds to the feeding operation, and the portion below 0 corresponds to the intake operation. The horizontal axis represents time. In this embodiment, the control unit 40 performs control so that the operation time of the feeding operation and the operation time of the intake operation of each plunger pump become equal. The control unit 40 also performs control so that only one of the two plunger pumps constantly carries out the feeding operation. The operation control of the plunger pumps by the control unit 40 is carried out indirectly as the control unit 40 controls the driving of the pump drive units 35, 36.

FIG. 6B shows displacement of each plunger 33p, 34p in the case where each plunger pump 33, 34 carries out the operation shown in FIG. 6A. The state where the plungers 33p, 34p are pulled out to the maximum from the syringes 33s, 34s corresponds to “0” on the vertical axis of the graph of FIG. 6B. The state where the plungers 33p, 34p are pushed to the maximum into the syringes 33s, 34s corresponds to “1” on the vertical axis of the graph of FIG. 6B. The vertices corresponding to maximum and minimum values on each graph curve corresponding to the syringes 33s, 34s are shown as gentler curves than the actual curves, in order to facilitate understanding of the explanation. However, the actual curves are steeper.

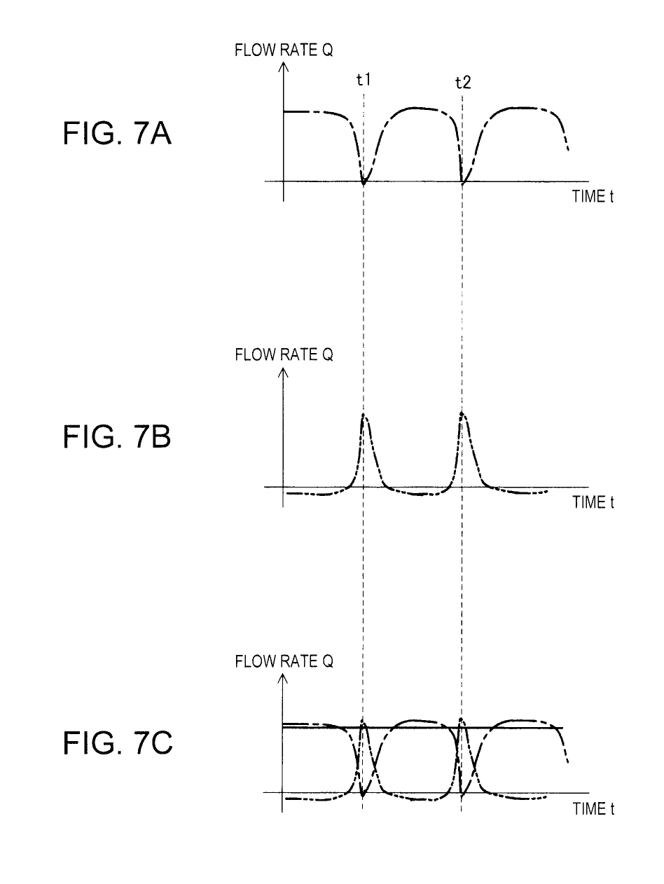

FIGS. 7A to 7C are explanatory views illustrating the pressure fluctuation restraint carried out by the control unit 40. FIG. 7A shows how the flow rate of water in the flow passage 73 changes according to the operation of the two plunger pumps 33, 34. As illustrated, the flow rate of water supplied to the water jet knife 20 from the flow passage 73 decreases at timings (t1, t2) when each plunger pump 33, 34 starts the feeding operation. This is because the plunger moving speed falls around the time when the feeding operation and the intake operation of the plunger pumps are switched, thus reducing the feeding pressure of water from the plunger pump to the flow passage 73. Specifically, since the feeding pressure falls, the internal pressure in the flow passage 73 falls and the flow rate of water circulating through the flow passage 73 decreases.

Also, when the plunger pumps are driven in such a way that the rise time and fall time of fluid feeding become equal, as shown in FIG. 6A, the flow rate of water that actually flows through the flow passage reaches the minimum value at t1, t2, but the curve along time is not symmetrical about t1, t2 and the rise is delayed with respect to the fall, as shown in FIG. 7A. This is because, due to the influence of air bubbles in the flow passage and the elastic action of the flow passage or the like, the feeding pressure does not necessarily rise in proportion to the plunger moving speed and has a temporal delay.

FIG. 7B is an explanatory view showing an image of change in the flow rate in the flow passage 73 caused solely by the operation of the flow passage deforming mechanism 50 in the case where the control unit 40 controls the flow passage deforming mechanism 50 according to fluctuation in the internal pressure in the flow passage 73. In practice, since the flow passage deforming mechanism 50 operates according to the internal pressure fluctuation in the flow passage 73 caused by the fluid feeding from the plunger pumps 33, 34, the operation of the flow passage deforming mechanism 50 does not take place by itself. However, in order to facilitate understanding, an image of flow rate fluctuation in the flow passage 73 caused solely by the operation of the flow passage deforming mechanism 50 according to the internal pressure fluctuation in the flow passage 73 is shown.

As illustrated, the control unit 40 controls the flow passage deforming mechanism 50 to press the flow passage 73 according to a reduction in the internal pressure detected by the pressure fluctuation detecting unit 60 and thus causes the internal pressure in the flow passage 73 to rise. As a result, the flow rate in the flow passage 73 (FIG. 7B) provided solely by the operation of the flow passage deforming mechanism 50 rises according to the reduction in the flow rate of water in the flow passage 73 caused by the two plunger pumps 33, 34, as shown in FIG. 7B.

Also, it can be seen that the change in the flow rate of water in the flow passage 73 caused solely by the flow passage deforming mechanism 50 is not symmetrical about the timings t1, t2 but is steep during the rise and gentle during the fall, as shown in FIG. 7B.

FIG. 7C shows a flow rate in the case where the change in the flow rate in the flow passage 73 due to the operation of the plunger pumps 33, 34, and the change in the flow rate in the flow passage 73 due to the operation of the flow passage deforming mechanism 50 are superimposed. That is, the illustrated graph shows the change in the flow rate in the flow passage 73 when the fluid supply apparatus 30 is actually supplying water to the water jet knife 20. The chain dotted line on the graph shows the change in the flow rate in the flow passage 73 due to the operation of the plunger pumps 33, 34. The double-chain dotted line shows the change in the flow rate in the flow passage 73 due to the operation of the flow passage deforming mechanism 50. The solid line shows a flow rate obtained by superimposing these two flow rates. As illustrated, it can be seen that fluctuation in the flow rate as a result of the superimposition is restrained. That is, the pressurization of the flow passage 73 by the flow passage deforming mechanism 50 compensates for the amount of fall in the pressure of water in the flow passage 73 due to the operation of the plunger pumps 33, 34. Since the change in the flow rate of water in the flow passage 73 caused solely by the operation of the flow passage deforming mechanism 50 reaches the maximum at the timings t1, t2 and is steep during the rise and gentle during the fall, this change is in a compensatory relation with the change in the flow rate shown in FIG. 7A, which reaches the minimum at the timings t1, t2 when the pumps are switched, and which is steep during the fall and gentle during the rise. When the two changes in the flow rate are superimposed, water flows through the flow passage 73 at a substantially constant flow rate having little change with time. Since the fluctuation in the pressure in the flow passage 73 is restrained, the fluctuation in the flow rate of water in the flow passage 73 is restrained. In this manner, as pressure fluctuation in the flow passage 73 due to the operation of the plunger pumps 33, 34 is detected by the pressure fluctuation detecting unit 60, the control unit 40 controls the operation of the flow passage deforming mechanism 50 according to the pressure fluctuation and thus restrains the pressure fluctuation in the internal pressure in the flow passage 73.

### A3. Water Jet Knife

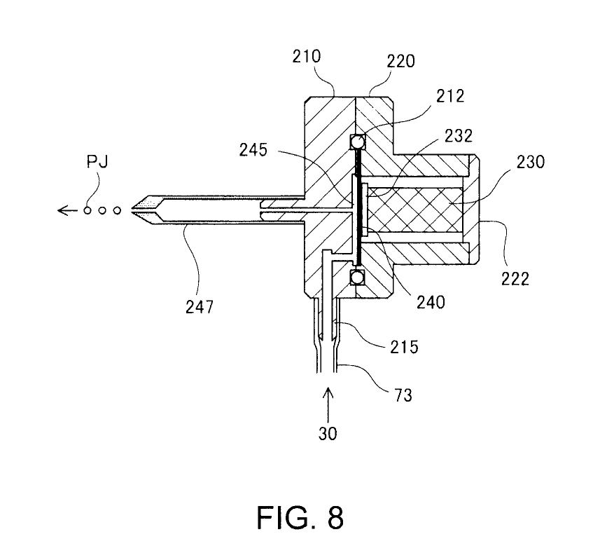

Next, the water jet knife 20 will be described. FIG. 8 is an explanatory view illustrating the structure of the water jet knife 20. The water jet knife 20 has an upper case 210, a lower case 220, a bottom portion 222, a piezoelectric element 230, an upper plate 232, a diaphragm 240, a packing 212, and a nozzle 247. The upper case 210 and the lower case 220 are joined together, facing each other. The lower case 220 is a cylindrical member and one end thereof is airtightly closed by the bottom portion 222. As illustrated, the piezoelectric element 230 is arranged in an inner space of the lower case 220.

The piezoelectric element 230 is a multilayer piezoelectric element and forms an actuator. One end of the piezoelectric element 230 is fixed to the diaphragm 240 via the upper plate 232. The other end of the piezoelectric element 230 is fixed to the bottom portion 222. The diaphragm 240 is made of a disc-shaped metal thin film and a circumferential edge thereof is fixed to the lower case 220. A pump chamber 245 is formed between the diaphragm 240 and the upper case 210 and the volume thereof changes as the piezoelectric element 230 is driven.

In the upper case 210, a flow passage connecting portion 215 for connecting a flow passage is formed. The flow passage 73 is connected to the flow passage connecting portion 215. The water supplied from the fluid supply apparatus 30 is supplied to the pump chamber 245 via the flow passage 73 and the flow passage connecting portion 215. When the piezoelectric element 230 oscillates at a predetermined frequency, the volume of the pump chamber 245 changes via the diaphragm 240 and the stored water is pressurized. The pressurized water is ejected through the nozzle 247 attached to the upper case 210.

Oscillation control of the piezoelectric element 230 is carried out by a control unit (not shown) of the water jet knife 20. As the control unit controls the oscillation of the piezoelectric element 230, the water jet knife 20 can eject pulse jets PJ in various forms. Up to this point is the explanation of the configuration of the water jet knife 20.

As described above, the fluid supply apparatus 30 can restrain pressure fluctuation of water supplied to the water jet knife 20 by causing the flow passage deforming mechanism 50 to operate according to pressure fluctuation in the internal pressure in the flow passage 73. As a result, flow rate fluctuation of the water supplied to the water jet knife 20 can be restrained. The fluid supply apparatus 30 can restrain pressure fluctuation by a relatively simple method such as deforming a flow passage. The control unit 40 uses the pressure fluctuation detecting unit 60 to detect pressure fluctuation in the flow passage 73 in real time and causes the flow passage deforming mechanism 50 to operate accordingly. Therefore, for example, internal pressure fluctuation due to a touch by a person on the flow passage, or pressure fluctuation due to various external factors such as mechanical vibration transmitted from a peripheral device can be dealt with and restrained. Moreover, it is also possible to carry out the pressure fluctuation restraint with respect to pressure fluctuation detected during a predetermined period only. For example, it is possible to detect pressure fluctuation generated in the flow passage 73 and carry out the pressure fluctuation restraint, only during a period when pressure fluctuation due to the operation of the plunger pumps is expected to occur.

In the water jet knife system 10, the flow passage 73 into which the flow passage 71 and the flow passage 72 join together is pressed by the flow passage deforming mechanism 50, thus restraining pressure fluctuation. Therefore, pressure fluctuation can be restrained simply by pressing a part of the flow passage. As a result, the pressure fluctuation restraint can be realized simply by providing one flow passage deforming mechanism 50. Thus, simplified control and structure and reduced cost can be realized.

Also, in this embodiment, a water jet knife is employed as a medical apparatus to which the fluid supply apparatus 30 supplies a fluid. In the water jet knife, supply of water with a stable flow rate is required. Therefore, by employing the fluid supply apparatus 30 as a fluid supply apparatus for supplying a fluid to the water jet knife, it is possible to supply water under stable pressure and at a stable flow rate, and characteristics of the fluid supply apparatus 30 can be exhibited to the maximum.

### B. Second Embodiment

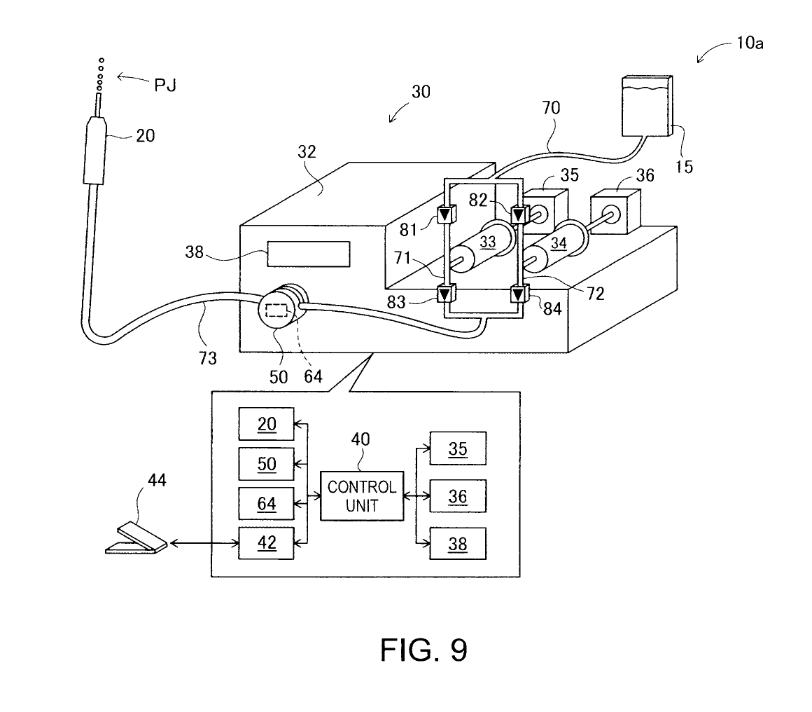

A second embodiment of the invention will be described. FIG. 9 is an explanatory view showing a water jet knife system 10a as a second embodiment. This embodiment is different from the first embodiment in that the fluid supply apparatus 30 has a pressure fluctuation detecting unit 64 inside the flow passage deforming mechanism 50, instead of the pressure fluctuation detecting unit 60. Thus, as illustrated, the control unit 40 is connected to the pressure fluctuation detecting unit 64 and controls the operation thereof.

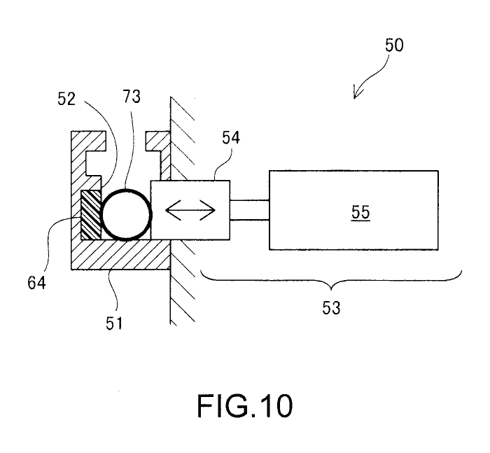

FIG. 10 is a vertical sectional view of the flow passage deforming mechanism 50 including the pressure fluctuation detecting unit 64. In this embodiment, the pressure fluctuation detecting unit 64 is formed as a load cell which measures a load. As illustrated, the pressure fluctuation detecting unit 64 faces the pressing portion 54 via the flow passage 73 and is installed in such a way that the pressure fluctuation detecting unit 64 and the pressing portion 54 hold the flow passage 73 from both sides. The pressure fluctuation detecting unit 64 is constantly in contact with the flow passage 73 and measures a force (load) received from the flow passage 73.

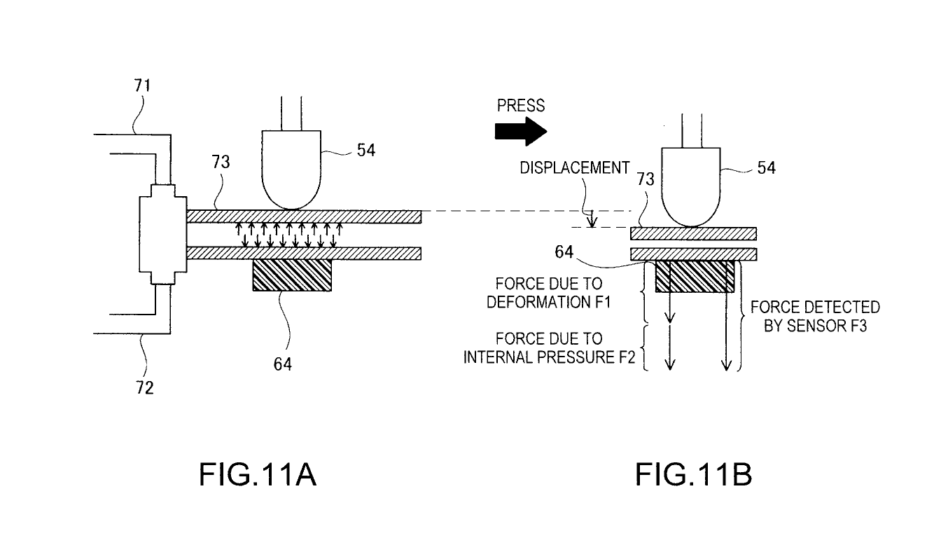

FIGS. 11A and 11B are explanatory views illustrating the force received by the pressure fluctuation detecting unit 64 (load cell) from the flow passage 73. FIG. 11A shows the state where the flow passage deforming mechanism 50 is not in operation. FIG. 11B shows the state where the flow passage deforming mechanism 50 is in operation, with the pressing portion 54 pressing the flow passage 73. As shown in FIG. 11B, when the flow passage deforming mechanism 50 is made to operate and the pressing portion 54 presses the flow passage 73, the pressure fluctuation detecting unit 64 receives a resultant force F3 of a force F1 due to the deformation of the flow passage and an internal pressure F2 in the flow passage 73. Therefore, the control unit 40 can subtract the force F1 due to the deformation of the flow passage from the resultant force F3 measured by the pressure fluctuation detecting unit 64, to acquire the internal pressure F2 in the flow passage 73. That is, the relation of F2=F3−F1 holds.

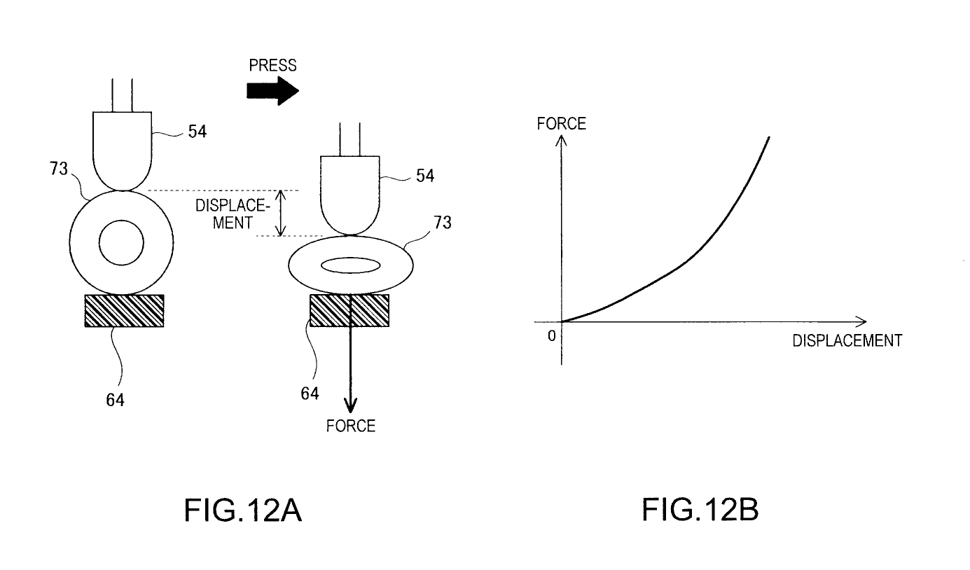

FIGS. 12A and 12B are explanatory views illustrating the force F1 due to the deformation of the flow passage. FIGS. 12A and 12B show the result of measuring the correlation between displacement and the force due to the deformation of the flow passage (corresponding to F1) in the case where the flow passage 73 is deformed as an independent component. That is, the graph shown in FIG. 12B represents characteristics of the flow passage 73 as an independent component. FIG. 12A shows the state at the time of measurement. In this embodiment, the flow passage 73 is fitted in the flow passage deforming mechanism 50 and the correlation is measured by using the flow passage deforming mechanism 50 and the pressure fluctuation detecting unit 64 in the state where the flow passage 73 is not supplied with water. FIG. 12B shows the result of the measurement.

The correlation between displacement and the force due to the deformation of the flow passage, thus measured on the flow passage 73 as an independent component, is provided as a lookup table in advance in the control unit 40. When the fluid supply apparatus 30 is in operation, the “displacement of the flow passage” corresponds to the amount of pushing by which the flow passage deforming mechanism 50 pushes the flow passage 73 in. Therefore, the control unit 40 can enter the amount of pushing by the flow passage deforming mechanism 50 into the lookup table, to acquire the force F1 due to the deformation of the flow passage. The control unit 40 can subtract the force F1 due to the deformation of the flow passage from the resultant force F3 measured by the pressure fluctuation detecting unit 64, to acquire the value of only the internal pressure F2 in the flow passage 73 in the case where the flow passage deforming mechanism 50 presses the flow passage 73.

When the fluid supply apparatus 30 is in operation, the control unit 40 acquires the internal pressure F2 at predetermined intervals. Then, the control unit 40 performs feedback control of the amount of pushing by the flow passage deforming mechanism 50 so that the value of the internal pressure F2 becomes constant, and thus restrains fluctuation in the internal pressure in the flow passage 73. In the second embodiment, the control unit 40 carries out pressure fluctuation restraint in this way.

As described above, since the water jet knife system 10a has the pressure fluctuation detecting unit 64 inside the flow passage deforming mechanism 50 instead of the pressure fluctuation detecting unit 60, the structure of the fluid supply apparatus 30 can be simplified and reduced in size. Also, since the position where pressure fluctuation is detected is the same as the position where the fluctuation is restrained, the pressure fluctuation restraint can be controlled without having to consider the time difference between the timing of detecting pressure fluctuation and the timing of restraining the pressure fluctuation.

### C. Modifications

The invention is not limited to the above embodiments and can be carried out in various embodiments without departing from the scope of the invention. For example, the following modifications are possible.

### C1. Modification 1

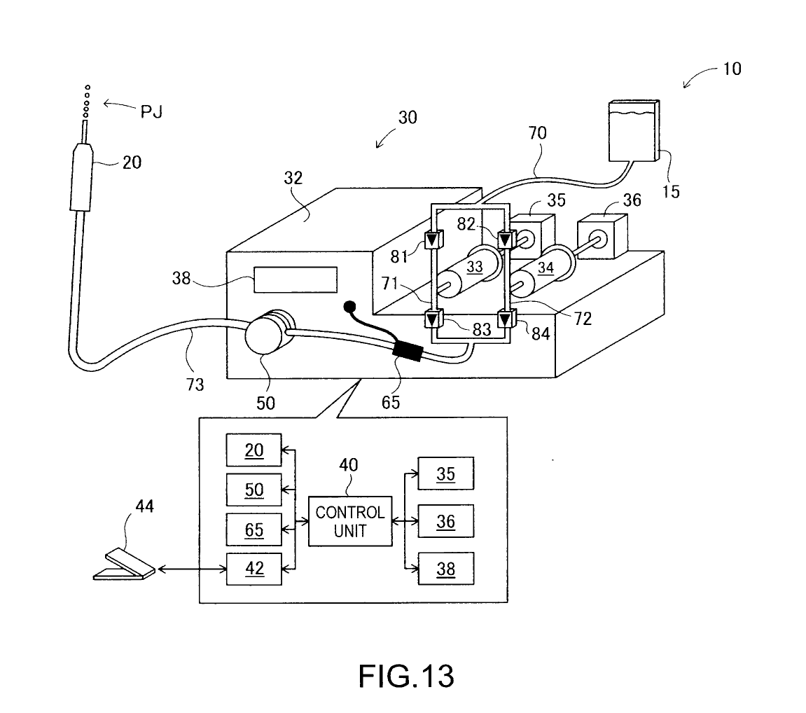

In the above embodiments, the pressure fluctuation detecting unit 60 and the pressure fluctuation detecting unit 64 are employed as a pressure fluctuation detecting unit for detecting fluctuation in the internal pressure in the flow passage. However, various other configurations can be employed without being limited to the above. FIG. 13 is an explanatory view showing a configuration in which the fluid supply apparatus 30 has a strain gauge 65 as a pressure fluctuation detecting unit, as an example of Modification 1. As illustrated, the strain gauge 65 is pasted and thus installed on the flow passage 73. The amount of strain of the flow passage 73 detected by the strain gauge 65 is converted to the amount of fluctuation in internal pressure by the control unit 40.

Specifically, the correlation between the internal pressure in the flow passage 73 as an independent component and the amount of strain measured by the strain gauge 65 is measured in advance, and the control unit 40 has a lookup table corresponding to the correlation. Then, when water is supplied to the water jet knife 20, the amount of strain acquired from the strain gauge 65 is converted to the internal pressure by using the lookup table, and the internal pressure and the amount of pressure fluctuation in the flow passage 73 are acquired. The flow passage deforming mechanism 50 is made to operate based on the acquired amount of pressure fluctuation, thus realizing pressure fluctuation restraint. Such a configuration enables detection of pressure fluctuation inside the flow passage 73 with a simple structure. Moreover, reduction in cost can be realized.

Also, instead of the laser displacement meter 63 employed in the first embodiment, various sensors capable of measuring displacement of the flow passage can be employed, such as a CCD camera, optical sensor using infrared rays, or acoustic sensor. If a CCD camera is employed, the width (diameter) of the flow passage 73 can be measured by picking up an image of the flow passage 73 and detecting the contour (edge) of the flow passage 73. This configuration can also achieve similar effects to the above embodiments.

### C2. Modification 2

In the above embodiments, the fluid supply apparatus 30 has two plunger pumps. However, plunger pumps may be provided in an arbitrary number equal to two or greater, such as three or four, as long as these plunger pumps can be installed in the fluid supply apparatus 30. Also, the timing of the intake operation and the feeding operation of each of the plural plunger pumps is not limited to the timing described with reference to FIGS. 6A and 6B, and various timings can be employed. For example, the timings of the feeding operations of the plural plunger pumps may overlap each other. Even in such a case, the control unit 40 can cause the flow passage deforming mechanism 50 to operate according to fluctuation in the internal pressure in the flow passage 73, to supply water to the water jet knife 20 at a stable flow rate.

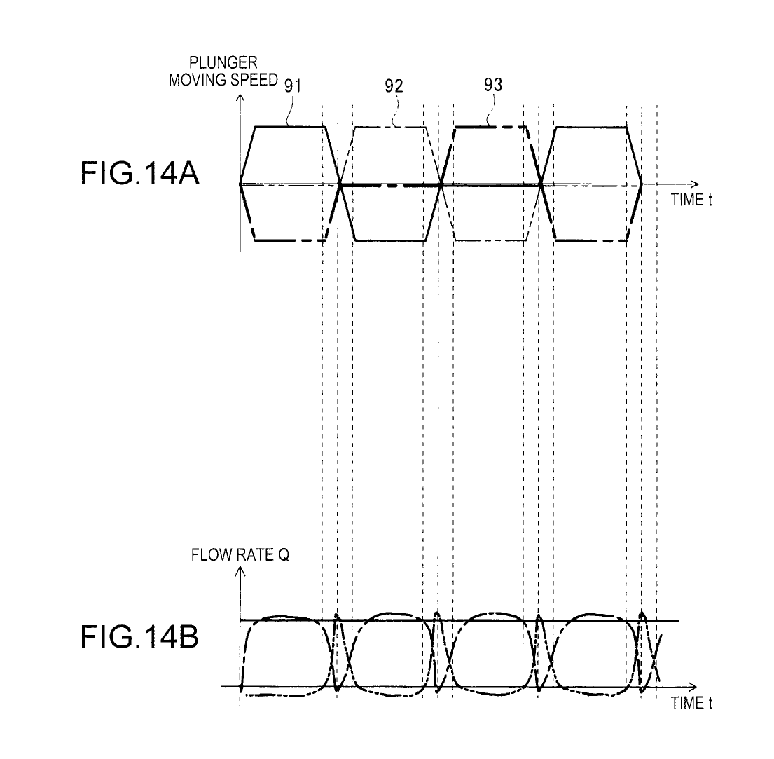

FIGS. 14A and 14B are explanatory views illustrating the case where the fluid supply apparatus 30 has three plunger pumps 91, 92, 93, as an example of Modification 2. FIG. 14A shows the moving speed of the plunger pumps 91, 92, 93. As illustrated, at any time, only one of the respective plunger pumps caries out the feeding operation. Each plunger pump carries out the feeding operation following a standby state after the intake operation.

FIG. 14B shows change in flow rate in the flow passage when the fluid supply apparatus 30 actually supplies water to the water jet knife 20. The chain dotted line on the graph shows change in the flow rate due to the operation of the plunger pumps 91, 92, 93. The double-chain dotted line shows change in the flow rate due to the operation of the flow passage deforming mechanism 50. The solid line shows the flow rate in the case where these two flow rates are superimposed. As illustrated, it can be seen that fluctuation in the superimposed flow rate is restrained. As in the foregoing embodiments, the control unit 40 uses the pressure fluctuation detecting unit to detect fluctuation in the internal pressure in the flow passage 73 caused by the plunger pumps 91, 92, 93 and causes the flow passage deforming mechanism 50 to operate according to the pressure fluctuation. Thus, similar effects to the foregoing embodiments can be achieved.

### C3. Modification 3

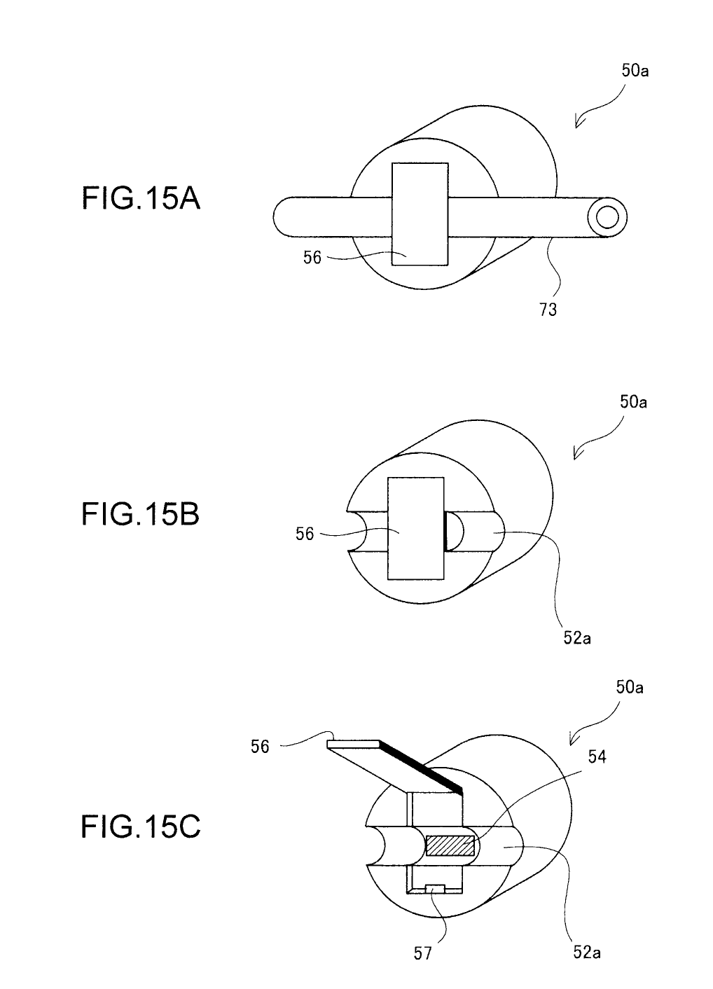

In the above Embodiment 1, the flow passage deforming mechanism 50 (FIGS. 3A and 3B, FIGS. 4A and 4B) is employed as a flow passage deforming unit. However, various forms can be employed without being limited to Embodiment 1, as long as the internal pressure in the flow passage can be changed by deformation of the flow passage. FIGS. 15A to 15C are explanatory views showing a flow passage deforming mechanism 50a as an example. The flow passage deforming mechanism 50a holds and fixes the flow passage 73 between a groove portion 52a and a movable portion 56 (see FIGS. 15A, 15B and 15C). As shown in FIG. 15C, the movable portion 56 is an open/close type. When the movable portion 56 is closed, a lock mechanism 57 (FIG. 15C) locks the movable portion 56.

As shown in FIG. 15C, the flow passage deforming mechanism 50a has a pressing portion 54a and presses the flow passage 73 under the control of the control unit 40. The drive mechanism of the pressing portion 54a is the same as the pressing mechanism 53 (FIGS. 4A and 4B) in the first embodiment and therefore will not be described further in detail. Such a configuration enables the flow passage 73 to be easily taken out of the flow passage deforming mechanism 50a even if the fluid supply apparatus 30 is stopped (for example, when power failure occurs) in the state where the flow passage deforming mechanism 50a is pressing the flow passage 73. In this way, the flow passage deforming unit can take various forms. Also, the way of deforming the flow passage 73 is not limited to pressing. Various forms of deformation to change the pressure inside the flow passage such as expansion/contraction or bending of the flow passage can be employed.

### C4. Modification 4

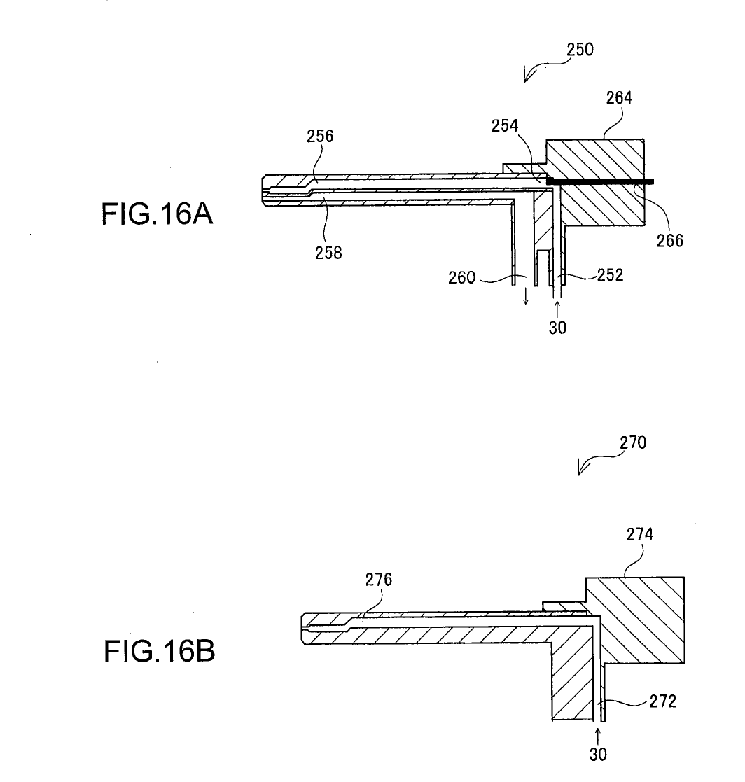

In the foregoing embodiments, the form of the water jet knife 20 described with reference to FIG. 8 is employed as a water jet knife. However, various forms of water jet knife may be employed without being limited to the embodiments. FIGS. 16A and 16B are explanatory views illustrating an example of a water jet knife that can be employed. FIG. 16A is an explanatory view illustrating the configuration of a water jet knife 250 utilizing a pulse laser. The water jet knife 250 has a intake path 252 for sucking in water from the fluid supply apparatus 30 via the flow passage 73, an air bubble generating unit 254 which generates air bubbles in the water that is sucked in, and an discharge flow passage 256 for ejecting the water. The water jet knife 250 also has a grip portion 264 for the user to hold the water jet knife 250, and an optical fiber 266.

The optical fiber 266 penetrates the grip portion 264 between the air bubble generating unit 254 and the outside. The optical fiber 266 extends outside of the grip portion 264 and is connected to a laser source (not shown). As a laser source, for example, a holmium-YAG laser (Ho-YAG laser: wavelength 2.1 μm) can be employed. The grip portion 264 supports the optical fiber 266 in the state where the distal end of the optical fiber 266 protrudes into the air bubble generating unit 254. The distal end of the optical fiber 266 protruding into the air bubble generating unit 254 is a pulse laser emission surface.

The water supplied from the fluid supply apparatus 30 circulates the flow passage 73 and the intake path 252, then fills the air bubble generating unit 254, and is ejected outward via the air bubble generating unit 254 and the discharge flow passage 256. As a pulse laser is emitted into the water from the distal end of the optical fiber 266 in the state where the air bubble generating unit 254 is filled with the water, the water absorbs the energy of the pulse laser and vaporizes instantaneously. Vapor bubbles are generated in the air bubble generating unit 254. With this generation of vapor bubbles, the internal pressure in the discharge flow passage 256 rises quickly and the water inside the discharge flow passage 256 is ejected outward from the discharge flow passage 256 as a pulse jet. The discharge speed of the pulse jet thus ejected is 10 m/s to 80 m/s and is capable of excising tissues of human bodies or the like.

The water jet knife 250 also has a drain 260 connected to a intake pump (not shown). The drain 260 communicates with a intake flow passage 258. For example, in a surgical operation, the water ejected from the discharge flow passage 256 stays at the surgical site as a drain fluid. In this case, the water jet knife 250 can use the intake force of the intake pump to suck the drain fluid staying at the surgical site via the intake flow passage 258.

With such a configuration, the water jet knife 250 can control the pulse jet PJ by laser emission. Also, since the water jet knife 250 does not have a drive unit to pressurize water, the structure of the water jet knife 250 can be simplified.

FIG. 16B is an explanatory view illustrating a water jet knife 270 that can be employed as a water jet knife. The water jet knife 270 has a intake path 272 for sucking water supplied from the fluid supply apparatus 30 via the flow passage 73, and an discharge flow passage 276 for ejecting the sucked water. The distal end of the discharge flow passage 276 is narrower than the inner part thereof. Therefore, the pressure of the water supplied from the fluid supply apparatus 30 is raised in the discharge flow passage 276 and the water is ejected outward. Since the water jet knife 270 does not have a mechanism for generating a pulse jet, the structure thereof can be simplified and reduced in weight.

### C5. Modification 5

In the second embodiment, the resultant force F3 (F1+F2) is measured by the load cell in the form of the pressure fluctuation detecting unit 64, and the flow passage is pressed by the flow passage deforming mechanism 50. However, a piezoelectric element having both functions of measuring the resultant force F3 and pressing may be used to carry out these two functions with one piezoelectric element. Specifically, the flow passage deforming mechanism 50 has a piezoelectric element instead of the pressing mechanism 53. The piezoelectric element is in contact with the flow passage 73. The control unit 40 uses the piezoelectric element to measure the resultant force F3 received from the flow passage 73. The control unit 40 acquires the internal pressure F2 based on the resultant force F3. The control unit 40 causes the piezoelectric element to expand and contract to press the flow passage 73 so that the value of the internal pressure F2 becomes constant. By taking this measure, the fluid supply apparatus 30 can also restrain pressure fluctuation in the flow passage 73. Moreover, simplification of the structure, reduction in size, and reduction is cost can be realized.

### C6. Modification 6

In the foregoing embodiments, pressure fluctuation is restrained by deforming the flow passage 73. However, pressure fluctuation may be restrained by deforming the flow passage 71 and the flow passage 72 at plural sites. That is, the fluid supply apparatus 30 may have plural flow passage deforming units.

### C7. Modification 7

The flow passage deforming mechanism 50 is not limited to the function of pressing the flow passage 73 for the purpose of pressure fluctuation restraint and may also have the function of a stop valve. For example, when the user carries out an operation to stop discharge of water from the water jet knife 20, the flow passage deforming mechanism 50 may press the flow passage 73 to block the flow passage under the control of the control unit 40 so that the circulation of water is completely stopped. Then, as the user carries out an operation to start discharge of water from the water jet knife 20, the flow passage deforming mechanism 50 may cancel the blocking of the flow passage 73 and let the water circulate under the control of the control unit 40. Thus, when discharge of water from the water jet knife 20 is stopped, leakage of water staying inside the flow passage from the nozzle of the water jet knife 20 can be restrained.

Also, the flow passage deforming mechanism 50 may have the function of a safety valve. For example, when the user wants to stop the supply of water urgently without waiting for the operation of the plunger pumps to stop, the flow passage deforming mechanism 50 may press and block the flow passage 73 to stop the supply of water. In this case, an operation unit for causing the flow passage deforming mechanism 50 to block the flow passage 73 may be provided as an emergency stop switch. Thus, the supply of water can be stopped immediately.

The blocking of the flow passage 73 by the flow passage deforming mechanism 50 may also function as a safety lock. The user is to carry out a water discharge operation by first carrying out a flow passage block canceling operation and then an discharge operation of the water jet knife 20. Thus, the fluid supply apparatus 30 can start ejecting water from the water jet knife 20 after making the user aware of the discharge of water.

### C8. Modification 8

In the foregoing embodiments, plunger pumps that carry out the feeding operation and the intake operation are employed as pumps. However, a pump that only carries out the feeding operation may be employed. The control unit 40 drives the flow passage deforming mechanism 50 according to the pressure fluctuation in the flow passage 73 detected by the pressure fluctuation detecting unit and restrains the pressure fluctuation in the flow passage. By taking this measure, fluctuation in the flow rate of water supplied can also be restrained.

### C9. Modification 9

In the foregoing embodiments, water is employed as a fluid. However, the fluid to be used is not limited to this. Various fluids, for example, physiological saline solution and low-viscosity oil, can be employed.

### C10. Modification 10

In the foregoing embodiments, a water jet knife is employed as a medical apparatus. However, the medical apparatus to be used is not limited to this. Various medical apparatuses, for example, a cleaner for cleaning an effected part in a surgical operation or treatment, and an apparatus for injecting a medical fluid into the body, can be employed.

### C11. Modification 11

In the foregoing embodiments, when the internal pressure in the flow passage is reduced, the internal pressure is raised by pressing the flow passage 73 as pressure fluctuation restraint. However, the pressure fluctuation restraint to be carried out is not limited to this and may be reducing the internal pressure when the internal pressure in the flow passage rises. Specifically, control may be performed to ease or cancel the pressing on the flow passage 73 which is constantly pressed at the time of fluid supply. By doing so, a pressure rise as pressure fluctuation can be restrained.

What is claimed is:

1. A fluid supply apparatus comprising:
a flow passage communicating with a medical apparatus;
a first pump capable of switching between intake of a fluid and discharge of the fluid to the flow passage;
a second pump capable of switching between intake of the fluid and discharge of the fluid to the flow passage;
a pressure fluctuation detecting unit capable of detecting fluctuation in internal pressure in the flow passage; and
a flow passage deforming unit which deforms a part of the flow passage according to the internal pressure detected by the pressure fluctuation detecting unit.

2. The fluid supply apparatus according to claim 1, wherein the pressure fluctuation detecting unit detects fluctuation in the internal pressure based on a displacement of an outer wall surface of the flow passage.

3. The fluid supply apparatus according to claim 1, wherein when the first pump ejects the fluid to the flow passage, the second pump sucks in the fluid,
when the first pump sucks in the fluid, the second pump ejects the fluid to the flow passage, and
the pressure fluctuation detecting unit detects fluctuation in the internal pressure in the flow passage when the first pump shifts from intake to discharge.

4. The fluid supply apparatus according to claim 1, wherein the pressure fluctuation detecting unit is installed on an outer wall surface of the flow passage, detects a force received from the flow passage, and detects fluctuation in the internal pressure based on a result of the detection.

5. The fluid supply apparatus according to claim 4, wherein the flow passage deforming unit has a piezoelectric element which deforms the flow passage, and
the pressure fluctuation detecting unit detects a force received from the flow passage, using the piezoelectric element.

6. The fluid supply apparatus according to claim 4, wherein the pressure fluctuation detecting unit is a strain gauge installed at a predetermined position on the outer wall surface of the flow passage.

7. The fluid supply apparatus according to claim 1, wherein the flow passage deforming unit is capable of blocking the flow passage by the deformation of the flow passage and thus stopping supply of the fluid.

8. The fluid supply apparatus according to claim 1, wherein the flow passage is disposable.

9. The fluid supply apparatus according to claim 1, wherein the medical apparatus is a therapeutic apparatus which ejects a fluid to a living body and thus treats the living body.

10. A control method for a fluid supply apparatus which supplies a fluid to a medical apparatus, the fluid supply apparatus including
a first pump which alternately carries out intake of the fluid and discharge to a flow passage communicating with the medical apparatus,
a second pump which alternately carries out intake of the fluid and discharge to the flow passage, and
a flow passage deforming unit which deforms a part of the flow passage,
the method comprising:
causing the second pump to suck in the fluid when the first pump ejects the fluid to the flow passage;
causing the second pump to eject the fluid to the flow passage when the first pump sucks in the fluid; and
causing the flow passage deforming unit to deform a part of the flow passage when the first pump shifts from intake to discharge.

11. The control method for the fluid supply apparatus according to claim 10, wherein the first pump and the second pump are plunger pumps, and
after a moving speed of a plunger of the first pump is raised during a predetermined period, the moving speed of the plunger of the first pump is lowered during a predetermined period.

12. The control method for the fluid supply apparatus according to claim 10, wherein the first pump and the second pump are plunger pumps,
after a moving speed of a plunger of the first pump is raised during a first period, the moving speed of the plunger of the first pump is lowered during a second period, and
after a moving speed of a plunger of the second pump is raised during the second period, the moving speed of the plunger of the second pump is lowered during the first period.

13. The control method for the fluid supply apparatus according to claim 10, wherein the first pump and the second pump are plunger pumps, and
when a moving speed of a plunger of the first pump rises, a moving speed of a plunger of the second pump falls.

14. The control method for the fluid supply apparatus according to claim 10, wherein the first pump and the second pump are plunger pumps, and
when a moving speed of a plunger of the second pump rises, a moving speed of a plunger of the first pump falls.
